(19) United States
(12) Patent Application Publication
(10) Pub. No.: US 2015/0218643 A1
(43) Pub. Date: Aug. 6, 2015

(54) DIFFERENTIAL METHYLATION LEVEL OF CPG LOCI THAT ARE DETERMINATIVE OF KIDNEY CANCER

(71) Applicants: Brittany N. Lasseigne, Huntsville, AL (US); Richard M. Myers, Huntsville, AL (US); Devin Absher, Huntsville, AL (US); James D. Brooks, Standford, CA (US)

(72) Inventors: Brittany N. Lasseigne, Huntsville, AL (US); Richard M. Myers, Huntsville, AL (US); Devin Absher, Huntsville, AL (US); James D. Brooks, Standford, CA (US)

(21) Appl. No.: 14/174,542

(22) Filed: Feb. 6, 2014

(51) Int. Cl. C12Q 1/68

(52) U.S. Cl. CPC C12Q 1/6886; C12Q 2600/154; C12Q 2600/118; C12Q 2600/16

(57) ABSTRACT

The present disclosure provides for and relates to the identification of novel biomarkers for diagnosis and prognosis of kidney cancer. The biomarkers of the invention show altered methylation levels of certain CpG loci relative to normal kidney tissue, as set forth.

### STATEMENT OF GOVERNMENT INTEREST

The U.S. Government may have an interest in, or certain rights to, the subject matter of this disclosure as provided for by the terms of grant number TCGA 3U24CA126563-03S1 and TATRC Cancer W81XWH-10-1-0790.

### FIELD OF THE DISCLOSURE

The present invention relates to compositions and methods for cancer diagnosis, research and therapy, including but not limited to, cancer biomarkers. In particular, the present invention relates to methylation levels of certain CpG loci as prognostic and diagnostic markers for kidney cancer, including without limitation, clear cell renal cell carcinoma (“ccRCC”).

### BACKGROUND

The kidneys are a pair of organs on either side of the spine in the lower abdomen, and are part of the urinary tract. They make urine by removing wastes and extra water from the blood. The kidneys also make substances that help control blood pressure and the production of red blood cells.

In 2013, approximately 65,000 cases of renal cell carcinoma (“RCC”) will be diagnosed in the United States and 13,600 patients will die of the disease. RCC incidence is rising approximately 2-3% per year, in large part due to the increasing use of abdominal imaging. Nearly half of all renal tumors are discovered incidentally, 20% of small tumors (less than 4 cm) are benign, and there are no imaging features or biomarkers that distinguish benign from malignant disease. For cancers confined to the kidney, the standard of care is resection, with high 5-year survival rates. Survival rates are directly correlated with tumor stage and size, demonstrating the importance of early detection of lesions when the lesions are small. Following tumor resection, patients must be monitored for recurrence at regular intervals by imaging studies (usually CT scanning) therefore incurring significant radiation exposure with the attendant risks. Once metastatic, RCC is usually fatal, despite treatment with targeted therapies, although a small fraction of patients show durable responses to IL-2 immunotherapy.

RCC is classified into histological subtypes with distinct clinical and pathogenic features. ccRCC, the most clinically aggressive subtype, comprises 75% of cases and is characterized by inactivation of the von Hippel-Lindau (VHL) tumor suppressor gene, a regulator of oxygen sensing in the cell by regulation of HIF1α protein levels¹⁴. Papillary RCC or pRCC (10% of cases), commonly has trisomy of chromosomes 7 and 17 and may be less clinically aggressive than ccRCC. Chromophobe carcinomas (chRCC) are the least aggressive tumors and comprise 5% of cases. Additionally, less common RCC subtypes arise from various cells of the nephron and present diverse clinical behavior¹⁵. Given the histologic, molecular, genetic, and clinical diversity of RCC and its origin from different cell types in the nephron, biomarkers for use across the most common histologic subtypes types of RCC for detection or monitoring have not been reported.

Current diagnostic tools for kidney cancer lack the sensitivity and specificity required for the detection of very early lesions/tumors and diagnosis ultimately relies on advanced imaging technologies or an invasive biopsy. Once kidney cancer is diagnosed, there are no available prognostic markers for kidney cancer that provide information on how aggressively the tumor will grow. Therefore, more intrusive therapeutic routes are often chosen that result in a drastic reduction in the quality of life for the patient, even though the majority of kidney tumors are slow growing and non-aggressive. This ultimately leads to undue burden on the healthcare system and an unnecessary decrease in quality of life for the patient. The present invention addresses the need for the diagnosis and prognostic determination of kidney tumors through identification of specific genomic DNA methylation biomarkers that can lead to early diagnosis of kidney cancer.

DNA methyltransferases (also referred to as DNA methylases) transfer methyl groups from the universal methyl donor S-adenosyl methionine to specific sites on a DNA molecule. Several biological functions have been attributed to the methylated bases in DNA, such as the protection of the DNA from digestion by restriction enzymes in prokaryotic cells. In eukaryotic cells, DNA methylation is an epigenetic method of altering DNA that influences gene expression, for example during embryogenesis and cellular differentiation. The most common type of DNA methylation in eukaryotic cells is the methylation of cytosine residues that are 5′ neighbors of guanine (“CG” dinucleotides, also referred to as “CpGs”). DNA methylation regulates biological processes without altering genomic sequence. DNA methylation regulates gene expression, DNA-protein interactions, cellular differentiation, suppresses transposable elements, and X chromosome inactivation.

Improper methylation of DNA is believed to be the cause of some diseases such as Beckwith-Wiedemann syndrome and Prader-Willi syndrome. It has also been purposed that improper methylation is a contributing factor in many cancers. For example, de novo methylation of the Rb gene has been demonstrated in retinoblastomas. In addition, expression of tumor suppressor genes have been shown to be abolished by de novo DNA methylation of a normally unmethylated 5′ CpG island. Many additional effects of methylation are discussed in detail in published International Patent Publication No. WO 00/051639.

Methylation of cytosines at their carbon-5 position plays an important role both during development and in tumorigenesis. Recent work has shown that the gene silencing effect of methylated regions is accomplished through the interaction of methylcytosine binding proteins with other structural components of chromatin, which, in turn, makes the DNA inaccessible to transcription factors through histone deacetylation and chromatin structure changes. The methylation occurs almost exclusively in CpG dinucleotides. While the bulk of human genomic DNA is depleted in CpG sites, there are CpG-rich stretches, so-called CpG islands, which are located in promoter regions of more than 70% of all known human genes. Epigenetic silencing of tumor suppressor genes by hypermethylation of CpG islands is a very early and stable characteristic of tumorigenesis. Hypermethylation of CpG islands located in the promoter regions of tumor suppressor genes are now firmly established as the most frequent mechanisms for gene inactivation in cancers.

### BRIEF DESCRIPTION OF THE DRAWINGS

FIG. 1 shows a PAM diagnostic panel model for RCC.

FIG. 2 shows a PAM diagnostic panel model for ccRCC.

### SUMMARY

The present invention relates to the identification of novel biomarkers for diagnosis and prognosis of kidney cancer. The biomarkers of the invention are CpG loci that have altered methylation levels relative to normal kidney tissue, as set forth, for example, in Table 1.

In some embodiments of the invention, the methylation level of one or a plurality of biomarkers set forth in Table 1 is determined in a patient sample suspected of comprising kidney cancer cells; wherein altered methylation at the indicated biomarker is indicative of kidney cancer. In some embodiments, a plurality of biomarkers is evaluated for altered methylation.

In some embodiments the patient sample is a tumor biopsy. In other embodiments the patient sample is a convenient bodily fluid, for example a blood sample, urine sample, and the like.

### DETAILED DESCRIPTION

### Introduction

While the invention has been described with respect to a limited number of embodiments, those skilled in the art, having benefit of this disclosure, will appreciate that other embodiments can be devised which do not depart from the scope of the invention as disclosed here.

The present invention is based, in part, on the discovery that sequences in certain DNA regions are methylated in cancer cells, but not normal cells, or that methylation level at specific loci in kidney cancer patients have a different methylation level then the same loci in patients without kidney cancer. Specifically, the inventors have found that methylation of biomarkers within the DNA regions described herein (such as those identified in Table 1) are associated with kidney cancer.

In view of this discovery, the inventors have recognized that methods for detecting the biomarker sequences and DNA regions comprising the biomarker sequences as well as sequences adjacent to the biomarkers that contain CpG loci subsequences, methylation level of the DNA regions, and/or expression of the genes regulated by the DNA regions can be used to predict recurrence of cancer cells or to detect cancer cells. Detecting cancer cells allows for diagnostic tests that detect disease, assess the risk of contracting disease, determining a predisposition to disease, stage disease, diagnosis of disease, monitor disease, and/or prognostic biomarkers such as these methylation markers can be used to aid in the selection of treatment for a patient.

### DEFINITIONS

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al, Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990), which are incorporated herein by reference. Enzymatic reactions and purification techniques, if any, are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are those well known and commonly used in the art. Standard techniques can be used for chemical syntheses, chemical analyses, pharmaceutical preparation, formulation, and delivery, and treatment of patients.

The term “individual” or “patient” as used herein refers to any animal, including mammals, such as, but not limited to, mice, rats, other rodents, rabbits, dogs, cats, swine, cattle, sheep, horses, primates, or humans.

The term “in need of prevention” as used herein refers to a judgment made by a caregiver that a patient requires or will benefit from prevention. This judgment is made based on a variety of factors that are in the realm of a caregiver's expertise, and may include the knowledge that the patient may become ill as the result of a disease state that is treatable by a compound or pharmaceutical composition of the disclosure.

The term “in need of treatment” as used herein refers to a judgment made by a caregiver that a patient requires or will benefit from treatment. This judgment is made based on a variety of factors that are in the realm of a caregiver's expertise, and may include the knowledge that the patient is ill as the result of a disease state that is treatable by a compound or pharmaceutical composition of the disclosure.

“Methylation” refers to cytosine methylation at positions C5 or N4 of cytosine, the N6 position of adenine or other types of nucleic acid methylation. In vitro amplified DNA is unmethylated because in vitro DNA amplification methods do not retain the methylation pattern of the amplification template. However, “unmethylated DNA” or “methylated DNA” can also refer to amplified DNA whose original template was methylated or methylated, respectively.

The term “methylation level” as applied to a gene refers to whether one or more cytosine residues present in a CpG context have or do not have a methylation group. Methylation level may also refer to the fraction of cells in a sample that do or do not have a methylation group on such cytosines. Methylation level may also alternatively describe whether a single CpG di-nucleotide is methylated.

A “methylation-dependent restriction enzyme” refers to a restriction enzyme that cleaves or digests DNA at or in proximity to a methylated recognition sequence, but does not cleave DNA at or near the same sequence when the recognition sequence is not methylated. Methylation-dependent restriction enzymes include those that cut at a methylated recognition sequence (e.g., DpnI) and enzymes that cut at a sequence near but not at the recognition sequence (e.g., McrBC). For example, McrBC's recognition sequence is 5′ RmC (N40-3000) RmC 3′ where “R” is a purine and “mC” is a methylated cytosine and “N40-3000” indicates the distance between the two RmC half sites for which a restriction event has been observed. McrBC generally cuts close to one half-site or the other, but cleavage positions are typically distributed over several base pairs, approximately 30 base pairs from the methylated base. McrBC sometimes cuts 3′ of both half sites, sometimes 5′ of both half sites, and sometimes between the two sites. Exemplary methylation-dependent restriction enzymes include, e.g., McrBC, McrA, MrrA, BisI, GlaI and DpnI. One of skill in the art will appreciate that any methylation-dependent restriction enzyme, including homologs and orthologs of the restriction enzymes described herein, is also suitable for use in the present invention.

A “methylation-sensitive restriction enzyme” refers to a restriction enzyme that cleaves DNA at or in proximity to an unmethylated recognition sequence but does not cleave at or in proximity to the same sequence when the recognition sequence is methylated. Exemplary methylation-sensitive restriction enzymes are described in, e.g., McClelland et al., Nucleic Acids Res. 22(17):3640-59 (1994) and http://rebase.neb.com. Suitable methylation-sensitive restriction enzymes that do not cleave DNA at or near their recognition sequence when a cytosine within the recognition sequence is methylated include, e.g., Aat II, Aci I, Acl I, Age I, Alu I, Asc I, Ase I, AsiS I, Bbe I, BsaA I, BsaH I, BsiE I, BsiW I, BsrF I, BssH II, BssK I, BstB I, BstN I, BstU I, Cla I, Eae L, Eag L, Fau I, Fse I, Hha I, HinP1 I, HinC II, Hpa II, Hpy99 I, HpyCH4 IV, Kas I, Mbo I, Mlu I, MapA1 I, Msp I, Nae I, Nar I, Not I, Pml I, Pst I, Pvu I, Rsr II, Sac II, Sap I, Sau3A I, Sfl I, Sfo I, SgrA I, Sma I, SnaB I, Tsc I, Xma I, and Zra I. Suitable methylation-sensitive restriction enzymes that do not cleave DNA at or near their recognition sequence when an adenosine within the recognition sequence is methylated at position N.sup.6 include, e.g., Mbo I. One of skill in the art will appreciate that any methylation-sensitive restriction enzyme, including homologs and orthologs of the restriction enzymes described herein, is also suitable for use in the present invention. One of skill in the art will further appreciate that a methylation-sensitive restriction enzyme that fails to cut in the presence of methylation of a cytosine at or near its recognition sequence may be insensitive to the presence of methylation of an adenosine at or near its recognition sequence. Likewise, a methylation-sensitive restriction enzyme that fails to cut in the presence of methylation of an adenosine at or near its recognition sequence may be insensitive to the presence of methylation of a cytosine at or near its recognition sequence. For example, Sau3AI is sensitive (i.e., fails to cut) to the presence of a methylated cytosine at or near its recognition sequence, but is insensitive (i.e., cuts) to the presence of a methylated adenosine at or near its recognition sequence. One of skill in the art will also appreciate that some methylation-sensitive restriction enzymes are blocked by methylation of bases on one or both strands of DNA encompassing of their recognition sequence, while other methylation-sensitive restriction enzymes are blocked only by methylation on both strands, but can cut if a recognition site is hemi-methylated.

The terms “peptide,” “polypeptide,” and “protein” each refer to a molecule comprising two or more amino acid residues joined to each other by peptide bonds. These terms encompass, e.g., native and artificial proteins, protein fragments and polypeptide analogs such as muteins, variants, and fusion proteins of a protein sequence as well as post-translationally, or otherwise covalently or non-covalently, modified proteins.

The terms “polynucleotide” and “nucleic acid” are used interchangeably throughout and include DNA molecules (e.g., cDNA or genomic DNA), RNA molecules (e.g., mRNA, siRNA), analogs of the DNA or RNA generated using nucleotide analogs (e.g., peptide nucleic acids and non-naturally occurring nucleotide analogs), and hybrids thereof. The nucleic acid molecule can be single-stranded or double-stranded. In one embodiment, the nucleic acid molecules of the invention comprise a contiguous open reading frame encoding an antibody, or a fragment, derivative, mutein, or variant thereof, of the invention. The nucleic acids can be any length. They can be, for example, 5, 10, 15, 20, 25, 30, 35, 40, 45, 50, 75, 100, 125, 150, 175, 200, 250, 300, 350, 400, 450, 500, 750, 1,000, 1,500, 3,000, 5,000 or more nucleotides in length, and/or can comprise one or more additional sequences, for example, regulatory sequences, and/or be part of a larger nucleic acid, for example, a vector.

The terms “prevent”, “preventing”, “prevention” “suppress”, “suppressing” and “suppression” as used herein refer to administering a compound either alone or as contained in a pharmaceutical composition prior to the onset of clinical symptoms of a disease state so as to prevent any symptom, aspect or characteristic of the disease state. Such preventing and suppressing need not be absolute to be useful.

The term “therapeutically effective amount”, in reference to the treating, preventing or suppressing of a disease state, refers to an amount of a compound either alone or as contained in a pharmaceutical composition that is capable of having any detectable, positive effect on any symptom, aspect, or characteristics of the disease state/condition. Such effect need not be absolute to be beneficial.

The terms “treat”, “treating” and “treatment” as used herein refers to administering a compound either alone or as contained in a pharmaceutical composition after the onset of clinical symptoms of a disease state so as to reduce or eliminate any symptom, aspect or characteristic of the disease state. Such treating need not be absolute to be useful.

### DNA Methylation Level and Cancer

DNA methylation is a heritable, reversible and epigenetic change. Yet, DNA methylation has the potential to alter gene expression, which has profound developmental and genetic consequences. The methylation reaction involves flipping a target cytosine out of an intact double helix to allow the transfer of a methyl group from S adenosyl-methionine in a cleft of the enzyme DNA (cystosine-5)-methyltransferase to form 5-methylcytosine (5-mCyt). This enzymatic conversion is the most common epigenetic modification of DNA known to exist in vertebrates, and is essential for normal embryonic development.

The presence of 5-mCyt at CpG dinucleotides has resulted in a 5-fold depletion of this sequence in the genome during vertebrate evolution, presumably due to spontaneous deamination of 5-mCyt to T. Those areas of the genome that do not show such suppression are referred to as “CpG islands”. These CpG island regions comprise about 1% of vertebrate genomes and also account for about 15% of the total number of CpG dinucleotides. CpG islands are typically between 0.2 to about 1 kb in length and are located upstream of many housekeeping and tissue-specific genes, but may also extend into gene coding regions. Therefore, the methylation levels of cytosine residues within CpG islands in somatic tissues can modulate gene expression throughout the genome. Methylation levels of cytosine residues contained within CpG islands of certain genes has been inversely correlated with gene activity. Thus, methylation of cytosine residues within CpG islands in somatic tissue is generally associated with decreased gene expression and can affect a variety of mechanisms including, for example, disruption of local chromatin structure, inhibition of transcription factor-DNA binding, or by recruitment of proteins which interact specifically with methylated sequences indirectly preventing transcription factor binding. Despite a generally inverse correlation between methylation of CpG islands and gene expression, most CpG islands on autosomal genes remain unmethylated in the germline and methylation of these islands is usually independent of gene expression. Tissue-specific genes are usually unmethylated at the receptive target organs but are methylated in the germline and in non-expressing adult tissues. CpG islands of constitutively-expressed housekeeping genes are normally unmethylated in the germline and in somatic tissues. A recent study showed evidence that methylation status of CpGs located within 2000 base pairs of a gene's transcription start site is negatively correlated with gene expression. For CpGs within a gene body, the methylation status of CpGs not in CpG islands is positively correlated with gene expression, whereas CpGs in the gene body in CpG islands can both negatively and positively impact gene expression (Varley et al, 2013).

Abnormal methylation of CpG islands associated with tumor suppressor genes can cause altered gene expression. Increased methylation (hypermethylation) of such regions can lead to progressive reduction of normal gene expression resulting in the selection of a population of cells having a selective growth advantage. Conversely, decreased methylation (hypomethylation) of oncogenes can lead to modulation of normal gene expression resulting in the selection of a population of cells having a selective growth advantage. In some examples, hypermethylation and/or hypomethylation of one or more CpG dinucleotide is considered to be abnormal methylation.

### Biomarkers

The present disclosure provides biomarkers useful for the detection of kidney cancer, wherein the methlyation level of the biomarker is indicative of the presence of kidney cancer. In one embodiment, the methylation level is determined by a cytosine. In one embodiment, the biomarkers are associated with certain genes in an individual. In one embodiment, the biomarkers are associated with certain CpG loci. In one embodiment, the CpG loci may be located in the promoter region of a gene, in an intron or exon of a gene or located near the gene in a patient's genomic DNA. In an alternate embodiment, the CpG may not be associated with any known gene or may be located in an intergenic region of a chromosome. In some embodiments, the CpG loci may be associated with one or more than one gene.

In one embodiment, the gene associated with the biomarker is C21orf123. In one embodiment, the CpG loci are cg02706881 (i.e., SEQ ID. NO. 1).

In an alternate embodiment, the gene associated with the biomarker is WISP2. In one embodiment, the CpG locus is cg03562120 (i.e., SEQ ID NO. 2).

In an alternate embodiment, the gene associated with the biomarker gene is GGT6. In one embodiment, the CpG locus is cg04511534 (i.e., SEQ ID NO. 3).

In yet an alternate embodiment, the gene associated with the biomarker gene is PENK. In one embodiment, the CpG locus is cg04598121 (i.e., SEQ ID NO. 4).

In yet an alternate embodiment, the gene associated with the biomarker is MPO. In one embodiment, the CpG locus is cg04988978 (i.e., SEQ ID NO. 5).

In an alternate embodiment, the gene associated with the biomarker GIT1. In one embodiment, the CpG locus is cg05379350 (i.e., SEQ ID NO. 6).

In an alternate embodiment, the gene associated with the biomarker is KLK10. In one embodiment, the CpG locus is cg06130787 (i.e., SEQ ID NO. 7).

In an alternate embodiment, the gene associated with the biomarker is RTP1. In one embodiment, the CpG locus is cg08749917 (i.e., SEQ ID NO. 8).

In an alternate embodiment, the gene associated with the biomarker is CHI3L2. In one embodiment, the CpG locus is cg10045881 (i.e., SEQ ID NO. 9).

In an alternate embodiment, the gene associated with the biomarker is AQP9. In one embodiment, the CpG locus is cg11098259 (i.e., SEQ ID NO. 10).

In an alternate embodiment, the gene associated with the biomarker is LEP. In one embodiment, the CpG locus is cg12782180 (i.e., SEQ ID NO. 11).

In an alternate embodiment, the gene associated with the biomarker is SAA2. In one embodiment, the CpG locus is cg12907644 (i.e., SEQ ID NO. 12).

In an alternate embodiment, the gene associated with the biomarker is VWA7. In one embodiment, the CpG locus is cg12939547 (i.e., SEQ ID NO. 13).

In an alternate embodiment, the gene associated with the biomarker is PTHR1. In one embodiment, the CpG locus is cg13156411 (i.e., SEQ ID NO. 14).

In an alternate embodiment, the gene associated with the biomarker is TBX6. In one embodiment, the CpG locus is cg14370448 (i.e., SEQ ID NO. 15).

In an alternate embodiment, the gene associated with the biomarker is RIN1. In one embodiment, the CpG locus is cg14391855 (i.e., SEQ ID NO. 16).

In an alternate embodiment, the gene associated with the biomarker is ZIC1. In one embodiment, the CpG locus is cg14456683 (i.e., SEQ ID NO. 17).

In an alternate embodiment, the gene associated with the biomarker is SAA1. In one embodiment, the CpG locus is cg15484375 (i.e., SEQ ID NO. 18).

In an alternate embodiment, the gene associated with the biomarker is EBI3. In one embodiment, the CpG locus is cg16592658 (i.e., SEQ ID NO. 19).

In an alternate embodiment, the gene associated with the biomarker is NFAM1. In one embodiment, the CpG locus is cg17568996 (i.e., SEQ ID NO. 20).

In an alternate embodiment, the gene associated with the biomarker is SLC25A18. In one embodiment, the CpG locus is cg18003231 (i.e., SEQ ID NO. 21).

In an alternate embodiment, the gene associated with the biomarker is GGT6. In one embodiment, the CpG locus is cg22628873 (i.e., SEQ ID NO. 22).

In an alternate embodiment, the gene associated with the biomarker is OPRM1. In one embodiment, the CpG locus is cg22719623 (i.e., SEQ ID NO. 23).

In an alternate embodiment, the gene associated with the biomarker is ARHGEF2. In one embodiment, the CpG locus is cg23320056 (i.e., SEQ ID NO. 24).

In an alternate embodiment, the gene associated with the biomarker is CHI3L2. In one embodiment, the CpG locus is cg26366091 (i.e., SEQ ID NO. 25).

In an alternate embodiment, the gene associated with the biomarker is GPR132. In one embodiment, the CpG locus is cg26514492 (i.e., SEQ ID NO. 26).

In an alternate embodiment, the gene associated with the biomarker is NOD2. In one embodiment, the CpG locus is cg26954174 (i.e., SEQ ID NO.27).

In one embodiment, the methylation level of one (1) of the following CpG loci may be determined (by any method set forth herein) to determine whether an individual is or may be at a risk for kidney cancer: cg02706881, cg04598121, cg05379350, cg06130787, cg08749917, cg12782180, cg12907644, cg12939547, cg13156411, cg14456683, cg17568996, cg18003231, cg22628873, cg22719623, cg23320056 or cg26514492. In some aspects, the methylation level of two (2) or more or three (3) or more of the forgoing CpG loci may be determined (by any method set forth herein) to determine whether an individual is or may be at a risk for kidney cancer.

In one embodiment, the methylation level of one (1) of the following CpG loci may be determined (by any method set forth herein) to determine whether an individual is or may be at a risk for ccRCC: cg03562120, cg10045881, cg11098259, cg14370448, cg16592658, cg26366091 or cg26954174. In some aspects, the methylation level of two (2) or more or three (3) or more of the forgoing CpG loci may be determined (by any method set forth herein) to determine whether an individual is or may be at a risk for ccRCC.

In one embodiment, the methylation level of one (1) of the following CpG loci may be determined (by any method set forth herein) to determine whether an individual is or may be at a risk for ccRCC or kidney cancer: cg04511534, cg04988978, cg14391855 or cg15484375. In some aspects, the methylation level of two (2) or more or three (3) or more of the forgoing CpG loci may be determined (by any method set forth herein) to determine whether an individual is or may be at a risk for ccRCC or kidney cancer.

In some aspects, the methylation level of any one of the following biomarkers and associated genes may be determined (by any method set forth herein) to determine whether an individual is or may be at a risk for ccRCC: WISP2, CHI3L2, AQP9, TBX6, EBI3 or NOD2. In some aspects, the methylation level of two (2) or more or three (3) or more of the forgoing biomarkers be determined (by any method set forth herein) to determine whether a patient is or may be at a risk for ccRCC.

In some aspects, the methylation level of any one of the following biomarkers and associated genes may be determined (by any method set forth herein) to determine whether an individual is or may be at a risk for ccRCC or kidney cancer: GGT6, MPO, RIN1 or SAA1. In some aspects, the methylation level of two (2) or more or three (3) or more of the forgoing biomarkers be determined (by any method set forth herein) to determine whether a patient is or may be at a risk for ccRCC or kidney cancer.

In some aspects, the methylation level of any one of the following biomarkers and associated genes may be determined (by any method set forth herein) to determine whether an individual is or may be at a risk for kidney cancer: C21orf123, PENK, GIT1, KLK10, RTP1, LEP, SAA2, VWA7, PTHR1, ZIC1, NFAM1, SLC25A18, GGT6, OPRM1, OPRM1 or GPR132. In some aspects, the methylation level of two (2) or more or three (3) or more of the forgoing biomarkers be determined (by any method set forth herein) to determine whether a patient is or may be at a risk for kidney cancer.

In one embodiment, an increase in the methylation level of one or more of the following CpG loci is indicative of kidney cancer: cg02706881, cg04598121, cg08749917, cg12782180, cg12939547, cg13156411, cg14456683, cg17568996, cg18003231, cg22628870 and cg22719623,

In one embodiment, an increase in the methylation level of one or more of the following CpG loci is indicative of ccRCC or kidney cancer: cg04511534.

In one embodiment, a decrease in the methylation level of one or more of the following CpG loci is indicative of kidney cancer: cg05379350, cg06130787, cg12907644, cg23320056 and cg26514492.

In one embodiment decrease in the methylation level of one or more of the following CpG loci is indicative of ccRCC: cg03562120, cg10045881, cg11098259, cg14370448, cg16592658, cg26366091 and cg26954174.

In one embodiment, a decrease in the methylation level of one or more of the following CpG loci is indicative of ccRCC or kidney cancer: cg04988978, cg14391855 and cg15484375

Table 1 shows the CpG loci, their chromosomal position (if known), and the genes associated with the CpG loci:

TABLE 1
The biomarkers of the present disclosure. The “CpG loci”
column is the reference number provided by Illumina's ® Golden
Gate and Infinium ® Assays. The “position”
column are the genomic positions that correspond to the most
current knowledge of the human genome sequence, which is the
Human February 2009 assembly known as GRCh37/hg19. Additionally
the position of each sequence in hg18 is also provided. The
nucleotide sequences of the CpG loci in Table 1 are shown in
Table 2 as well as the sequence listing filed herewith. The
specific site of methylation is underlined in the nucleotide
sequence shown in Table 2.

| | | Associated | Position | Position | |
|---|---|---|---|---|---|
| | Chro- | Gene(s)/ | in Human | in Human | |
| CpG loci | mo- | Known | Genome | Genome | SEQ ID |
| Sequence | some | Function | 19 (hg19) | 18 (hg18) | NO. |
| cg02706881 | 21 | C21orf123 | 46845775 | 45670203 | SEQ ID |
| | | | | | NO. 1 |
| cg03562120 | 20 | WISP2 | 43343997 | 42777411 | SEQ ID |
| | | | | | NO. 2 |
| cg04511534 | 17 | GGT6 | 4463371 | 4410120 | SEQ ID |
| | | | | | NO. 3 |
| cg04598121 | 8 | PENK | 57358505 | 57521059 | SEQ ID |
| | | | | | NO. 4 |
| cg04988978 | 17 | MPO | 56359578 | 53714577 | SEQ ID |
| | | | | | NO. 5 |
| cg05379350 | 17 | GIT1 | 27917157 | 24941283 | SEQ ID |
| | | | | | NO. 6 |
| cg06130787 | 19 | KLK10 | 51523550 | 56215362 | SEQ ID |
| | | | | | NO. 7 |
| cg08749917 | 3 | RTP1 | 186915320 | 188398014 | SEQ ID |
| | | | | | NO. 8 |
| cg10045881 | 1 | CHI3L2 | 111770291 | 111571814 | SEQ ID |
| | | | | | NO. 9 |
| cg11098259 | 15 | AQP9 | 58430391 | 56217683 | SEQ ID |
| | | | | | NO. 10 |
| cg12782180 | 7 | LEP | 127880932 | 127668168 | SEQ ID |
| | | | | | NO. 11 |
| cg12907644 | 11 | SAA2 | 18270341 | 18226917 | SEQ ID |
| | | | | | NO. 12 |
| cg12939547 | 6 | VWA7 | 31744037 | 31852016 | SEQ ID |
| | | | | | NO. 13 |
| cg13156411 | 3 | PTHR1 | 46919454 | 46894458 | SEQ ID |
| | | | | | NO. 14 |
| cg14370448 | 16 | TBX6 | 30103978 | 30011479 | SEQ ID |
| | | | | | NO. 15 |
| cg14391855 | 11 | RIN1 | 66104174 | 65860750 | SEQ ID |
| | | | | | NO. 16 |
| cg14456683 | 3 | ZIC1 | 147127010 | 148609700 | SEQ ID |
| | | | | | NO. 17 |
| cg15484375 | 11 | SAA1 | 18287647 | 18244223 | SEQ ID |
| | | | | | NO. 18 |
| cg16592658 | 19 | EBI3 | 4229887 | 4180887 | SEQ ID |
| | | | | | NO. 19 |
| cg17568996 | 22 | NFAM1 | 42828125 | 41158069 | SEQ ID |
| | | | | | NO. 20 |
| cg18003231 | 22 | SLC25A18 | 18043745 | 16423745 | SEQ ID |
| | | | | | NO. 21 |
| cg22628873 | 17 | GGT6 | 4464400 | 4411149 | SEQ ID |
| | | | | | NO. 22 |
| cg22719623 | 6 | OPRM1 | 154360732 | 154402425 | SEQ ID |
| | | | | | NO. 23 |
| cg23320056 | 1 | OPRM1 | 155948742 | 154215366 | SEQ ID |
| | | | | | NO. 24 |
| cg26366091 | 1 | CHI3L2 | 111770274 | 111571797 | SEQ ID |
| | | | | | NO. 25 |
| cg26514492 | 14 | GPR132 | 105531893 | 104602938 | SEQ ID |
| | | | | | NO. 26 |
| cg26954174 | 16 | NOD2 | 50730813 | 49288314 | SEQ ID |
| | | | | | NO. 27 |

### Use of Biomarkers

In some embodiments, the methylation level of the chromosomal DNA within a DNA region or portion thereof (e.g., at least one cytosine residue) selected from the CpG loci identified in Table 1 is determined. In some embodiments, the methylation level of all cytosines within at least 20, 50, 100, 200, 500 or more contiguous base pairs of the CpG loci is also determined. For example, in one embodiment, the methylation level of the cytosine at cg15484375 is determined. In some embodiments, pluralities of CpG loci are assessed and their methylation level determined.

In some embodiments of the invention, the methylation level of a CpG loci is determined and then normalized (e.g., compared) to the methylation of a control locus. Typically the control locus will have a known, relatively constant, methylation level. For example, the control sequence can be previously determined to have no, some or a high amount of methylation (or methylation level), thereby providing a relative constant value to control for error in detection methods, etc., unrelated to the presence or absence of cancer. In some embodiments, the control locus is endogenous, i.e., is part of the genome of the individual sampled. For example, in mammalian cells, the testes-specific histone 2B gene (hTH2B in human) gene is known to be methylated in all somatic tissues except testes. Alternatively, the control locus can be an exogenous locus, i.e., a DNA sequence spiked into the sample in a known quantity and having a known methylation level.

The methylation sites in a DNA region can reside in non-coding transcriptional control sequences (e.g. promoters, enhancers, etc.) or in coding sequences, including introns and exons of the associated genes. In some embodiments, the methods comprise detecting the methylation level in the promoter regions (e.g., comprising the nucleic acid sequence that is about 1.0 kb, 1.5 kb, 2.0 kb, 2.5 kb, 3.0 kb, 3.5 kb or 4.0 kb 5′ from the transcriptional start site through to the transcriptional start site) of one or more of the associated genes identified in Table 1.

Any method for detecting methylation levels can be used in the methods of the present invention.

In some embodiments, methods for detecting methylation levels include randomly shearing or randomly fragmenting the genomic DNA, cutting the DNA with a methylation-dependent or methylation-sensitive restriction enzyme and subsequently selectively identifying and/or analyzing the cut or uncut DNA. Selective identification can include, for example, separating cut and uncut DNA (e.g., by size) and quantifying a sequence of interest that was cut or, alternatively, that was not cut. Alternatively, the method can encompass amplifying intact DNA after restriction enzyme digestion, thereby only amplifying DNA that was not cleaved by the restriction enzyme in the area amplified. In some embodiments, amplification can be performed using primers that are gene specific. Alternatively, adaptors can be added to the ends of the randomly fragmented DNA, the DNA can be digested with a methylation-dependent or methylation-sensitive restriction enzyme, intact DNA can be amplified using primers that hybridize to the adaptor sequences. In this case, a second step can be performed to determine the presence, absence or quantity of a particular gene in an amplified pool of DNA. In some embodiments, the DNA is amplified using real-time, quantitative PCR.

In some embodiments, the methods comprise quantifying the average methylation density in a target sequence within a population of genomic DNA. In some embodiments, the method comprises contacting genomic DNA with a methylation-dependent restriction enzyme or methylation-sensitive restriction enzyme under conditions that allow for at least some copies of potential restriction enzyme cleavage sites in the locus to remain uncleaved; quantifying intact copies of the locus; and comparing the quantity of amplified product to a control value representing the quantity of methylation of control DNA, thereby quantifying the average methylation density in the locus compared to the methylation density of the control DNA.

The methylation level of a CpG loci can be determined by providing a sample of genomic DNA comprising the CpG locus, cleaving the DNA with a restriction enzyme that is either methylation-sensitive or methylation-dependent, and then quantifying the amount of intact DNA or quantifying the amount of cut DNA at the locus of interest. The amount of intact or cut DNA will depend on the initial amount of genomic DNA containing the locus, the amount of methylation in the locus, and the number (i.e., the fraction) of nucleotides in the locus that are methylated in the genomic DNA. The amount of methylation in a DNA locus can be determined by comparing the quantity of intact DNA or cut DNA to a control value representing the quantity of intact DNA or cut DNA in a similarly-treated DNA sample. The control value can represent a known or predicted number of methylated nucleotides. Alternatively, the control value can represent the quantity of intact or cut DNA from the same locus in another (e.g., normal, non-diseased) cell or a second locus.

By using at least one methylation-sensitive or methylation-dependent restriction enzyme under conditions that allow for at least some copies of potential restriction enzyme cleavage sites in the locus to remain uncleaved and subsequently quantifying the remaining intact copies and comparing the quantity to a control, average methylation density of a locus can be determined. If the methylation-sensitive restriction enzyme is contacted to copies of a DNA locus under conditions that allow for at least some copies of potential restriction enzyme cleavage sites in the locus to remain uncleaved, then the remaining intact DNA will be directly proportional to the methylation density, and thus may be compared to a control to determine the relative methylation density of the locus in the sample. Similarly, if a methylation-dependent restriction enzyme is contacted to copies of a DNA locus under conditions that allow for at least some copies of potential restriction enzyme cleavage sites in the locus to remain uncleaved, then the remaining intact DNA will be inversely proportional to the methylation density, and thus may be compared to a control to determine the relative methylation density of the locus in the sample.

Kits for the above methods can include, e.g., one or more of methylation-dependent restriction enzymes, methylation-sensitive restriction enzymes, amplification (e.g., PCR) reagents, probes and/or primers.

Quantitative amplification methods (e.g., quantitative PCR or quantitative linear amplification) can be used to quantify the amount of intact DNA within a locus flanked by amplification primers following restriction digestion. Methods of quantitative amplification are disclosed in, e.g., U.S. Pat. Nos. 6,180,349; 6,033,854; and 5,972,602. Amplifications may be monitored in “real time.”

Additional methods for detecting methylation levels can involve genomic sequencing before and after treatment of the DNA with bisulfite. When sodium bisulfite is contacted to DNA, unmethylated cytosine is converted to uracil, while methylated cytosine is not modified. Such additional embodiments include the use of array-based assays such as the Illumina® Human Methylation450 BeadChip and multi-plex PCR assays. In one embodiment, the multi-plex PCR assay is PatchPCR. PatchPCR can be used to determine the methylation level of a certain CpG loci. See Varley KE and Mitra RD (2010). Bisulfite PatchPCR enables multiplexed sequencing of promoter methylation across cancer samples. Genome Research. 20:1279-1287.

In some embodiments, restriction enzyme digestion of PCR products amplified from bisulfite-converted DNA is used to detect DNA methylation levels.

In some embodiments, a “MethyLight” assay is used alone or in combination with other methods to detect methylation level. Briefly, in the MethyLight process, genomic DNA is converted in a sodium bisulfite reaction (the bisulfite process converts unmethylated cytosine residues to uracil). Amplification of a DNA sequence of interest is then performed using PCR primers that hybridize to CpG dinucleotides. By using primers that hybridize only to sequences resulting from bisulfite conversion of unmethylated DNA, (or alternatively to methylated sequences that are not converted) amplification can indicate methylation status of sequences where the primers hybridize. Similarly, the amplification product can be detected with a probe that specifically binds to a sequence resulting from bisulfite treatment of a unmethylated (or methylated) DNA. If desired, both primers and probes can be used to detect methylation status. Thus, kits for use with MethyLight can include sodium bisulfite as well as primers or detectably-labeled probes (including but not limited to Taqman or molecular beacon probes) that distinguish between methylated and unmethylated DNA that have been treated with bisulfite. Other kit components can include, e.g., reagents necessary for amplification of DNA including but not limited to, PCR buffers, deoxynucleotides; and a thermostable polymerase.

In some embodiments, a Ms-SNuPE (Methylation-sensitive Single Nucleotide Primer Extension) reaction is used alone or in combination with other methods to detect methylation level. The Ms-SNuPE technique is a quantitative method for assessing methylation differences at specific CpG sites based on bisulfite treatment of DNA, followed by single-nucleotide primer extension. Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site(s) of interest.

Typical reagents (e.g., as might be found in a typical Ms-SNuPE-based kit) for Ms-SNuPE analysis can include, but are not limited to: PCR primers for specific gene (or methylation-altered DNA sequence or CpG island); optimized PCR buffers and deoxynucleotides; gel extraction kit; positive control primers; Ms-SNuPE primers for a specific gene; reaction buffer (for the Ms-SNuPE reaction); and detectably-labeled nucleotides. Additionally, bisulfite conversion reagents may include: DNA denaturation buffer; sulfonation buffer; DNA recovery regents or kit (e.g., precipitation, ultrafiltration, affinity column); desulfonation buffer; and DNA recovery components.

In some embodiments, a methylation-specific PCR (“MSP”) reaction is used alone or in combination with other methods to detect DNA methylation. An MSP assay entails initial modification of DNA by sodium bisulfite, converting all unmethylated, but not methylated, cytosines to uracil, and subsequent amplification with primers specific for methylated versus unmethylated DNA.

Additional methylation level detection methods include, but are not limited to, methylated CpG island amplification and those described in, e.g., U.S. Patent Publication 2005/0069879; Rein, et al. Nucleic Acids Res. 26 (10): 2255-64 (1998); Olek, et al. Nat. Genet. 17(3): 275-6 (1997); and PCT Publication No. WO 00/70090.

### Kits

This invention also provides kits for the detection and/or quantification of the diagnostic biomarkers of the invention, or expression or methylation level thereof using the methods described herein.

The kits for detection of methylation level can comprise at least one polynucleotide that hybridizes to one of the CpG loci identified in Table 1 (or a nucleic acid sequence at least 90%, 92%, 95% and 97% identical to the CpG loci of Tale 1), or that hybridizes to a region of DNA flanking one of the CpG identified in Table 1, and at least one reagent for detection of gene methylation. Reagents for detection of methylation include, e.g., sodium bisulfite, polynucleotides designed to hybridize to sequence that is the product of a biomarker sequence of the invention if the biomarker sequence is not methylated, and/or a methylation-sensitive or methylation-dependent restriction enzyme. The kits can provide solid supports in the form of an assay apparatus that is adapted to use in the assay. The kits may further comprise detectable labels, optionally linked to a polynucleotide, e.g., a probe, in the kit. Other materials useful in the performance of the assays can also be included in the kits, including test tubes, transfer pipettes, and the like. The kits can also include written instructions for the use of one or more of these reagents in any of the assays described herein.

In some embodiments, the kits of the invention comprise one or more (e.g., 1, 2, 3, 4, or more) different polynucleotides (e.g., primers and/or probes) capable of specifically amplifying at least a portion of a DNA region where the DNA region includes one of the CpG Loci identified in Table 1. Optionally, one or more detectably-labeled polypeptides capable of hybridizing to the amplified portion can also be included in the kit. In some embodiments, the kits comprise sufficient primers to amplify 2, 3, 4, 5, 6, 7, 8, 9, 10, or more different DNA regions or portions thereof, and optionally include detectably-labeled polynucleotides capable of hybridizing to each amplified DNA region or portion thereof. The kits further can comprise a methylation-dependent or methylation sensitive restriction enzyme and/or sodium bisulfite.

### Methods of Diagnosis and Methods of Treatment

The present disclosure provides methods for the treatment and/or prevention of a disease state that is characterized, at least in part, by the altered methylation level of the CpG loci identified in Table 1.

In one embodiment, the altered methylation at CpG loci are associated with the occurrence in a patient of a cancer. In one embodiment, the cancer is kidney cancer. In a more specific embodiment, the kidney cancer is ccRCC. In one embodiment, the altered methylation levels of the CpG loci are associated with the reoccurrence of kidney cancer. In one embodiment, the altered methylation levels of the CpG loci is differentially diagnostic in a patient suffering from kidney cancer as compared to a patient not suffering from kidney cancer.

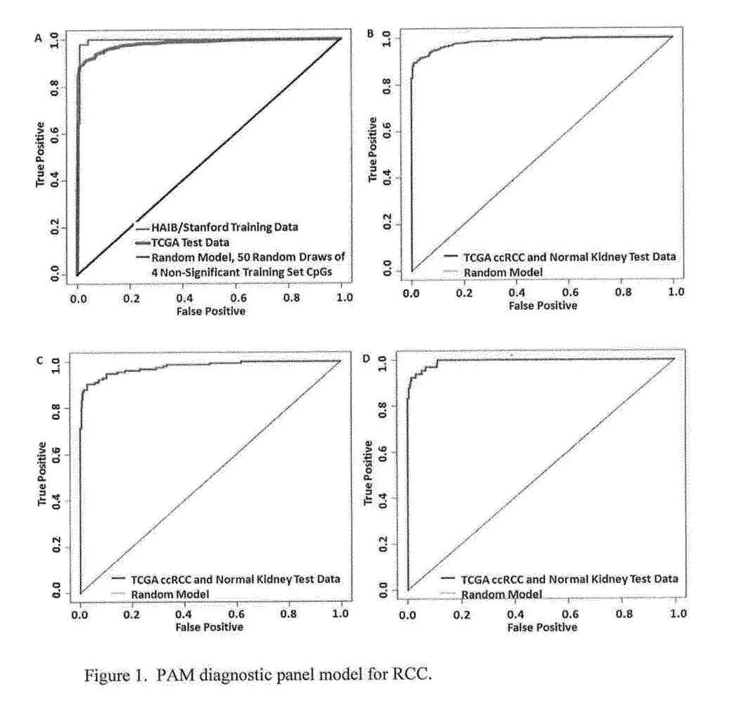

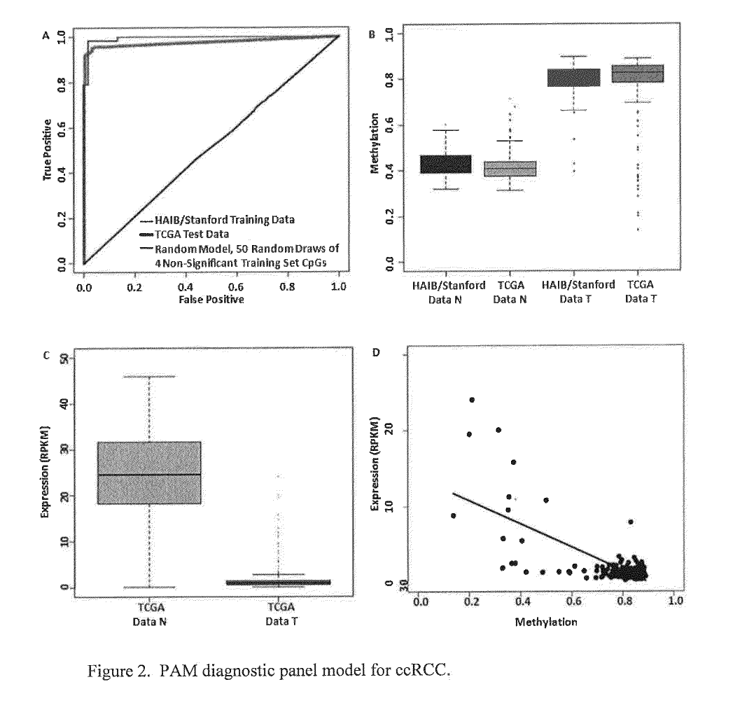

As illustrated in FIGS. 1 and 2, determining the methylation levels of at least one of the CpG loci identified in Table 1 is predictive of kidney cancer. FIG. 1 shows PAM diagnostic panel model for renal cell carcinoma. (A) ROC curve of best 5 CpG model (Benjamini and Hochberg adjusted p-value=8.10×10⁻³¹) from PAM diagnostic panel produced via the HAIB/Stanford data (ROC AUC=0.991), and applied to the TCGA data (ROC AUC=0.990). (B) ROC curve of best 5 CpG model applied to TCGA ccRCC and normal kidney tissue data (ROC AUC=0.98). (C) ROC curve of best 5 CpG model applied to TCGA pRCC and normal kidney tissue data (ROC AUC=0.97). (D) ROC curve of best 5 CpG model applied to TCGA chRCC and normal kidney tissue data (ROC AUC=0.99).

FIG. 2 shows the PAM diagnostic panel model for clear cell renal cell carcinoma. (A) ROC curve of best 4 CpG model (Benjamini and Hochberg adjusted p-value=1.46×10⁻²⁰) from PAM diagnostic panel produced in the HAIB/Stanford data (ROC AUC=0.990) and applied to the TCGA (ROC AUC=0.972). (B) DNA methylation at cg04511534, a CpG in the most predictive HAIB/Stanford model (Mann-Whitney test; Bonferroni adjusted p-value=0.2524 for HAIB/Stanford normals versus TCGA normals; Bonferroni adjusted p-value=0.1848 for HAIB/Stanford tumors versus TCGA tumors; Bonferroni adjusted p-value<0.0001 for HAIB/Stanford normal versus TCGA tumor, Bonferroni adjusted p-value<0.0001 for HAIB/Stanford tumor versus TCGA normal). (C) Expression of GGT6 in HAIB/Stanford tumor and normal tissue data (Mann-Whitney test; p-value<0.0001). (D) GGT6 expression versus cg04511534 methylation in TCGA tumor data (linear regression; p-value<0.0001, R²=0.5030).

Other non-limiting methods of diagnosis and treatment are described below. In this embodiment, the methylation levels of the CpG loci identified in Table 1 is detected to aid in the treatment, prevention or diagnosis of a cancer, such as kidney cancer.

The steps in the method of treatment or prevention, in one embodiment are:

A. Identifying a patient in need of the prevention or treatment of kidney cancer. This identifying step may be accomplished by many different methods. The patient could be identified by a physician who believes the patient would benefit from such treatment prevention or by standard genetic screening or analysis indicating the patient would benefit from such treatment or prevention.

B. Obtaining a sample from the patient. In some embodiments the patient sample is a tumor biopsy. In other embodiments the patient sample is a convenient bodily fluid, for example a blood sample, urine sample, and the like. The sample may be obtained by other means as well.

C. Determining the methylation levels of one or more of the CpG loci or dinculetides at the positions identified on Table 1. This determination step may be accomplished by any of the means set forth in this disclosure. In one embodiment, the methylation level of one of the CpG loci is determined while in other embodiments, the methylation levels of a plurality of the CpG loci are determined.

D. Comparing the methylation levels of CpG loci determined in step “C” to a reference or control. In one embodiment, a methylation level of the CpG loci determined in step “C” different from the control is indicative of presence of kidney cancer. This comparison step may be accomplished by any of the methods set forth herein.

E. Treating the patient with a therapeutically effective amount of a composition or radiation therapy if the comparing step in “D” above indicates the presence of kidney cancer. In one embodiment, the composition may include compounds for hormone therapy such as androgen deprivation therapy.

In an alternate embodiment, the present invention provides methods for determining the methylation status of an individual. In one aspect, the methods comprise obtaining a biological sample from an individual; and determining the methylation level of at least one cytosine within a DNA region in a sample from an individual where the DNA region is at least 90%, 91%, 92%, 93%, 94%, 95%, 96%, 97%, 98%, or 99% identical to, or comprises, a sequence selected from the group consisting of SEQ ID NOS.: 1-27.

In some embodiments, the methods comprise:
A. Determining the methylation status of at least one cytosine within a DNA region in a sample from the individual where the DNA region is at least 90%, 91%, 92%, 93%, 94%, 95%, 96%, 97%, 98%, or 99% identical to, or comprises, a sequence selected from the group consisting of SEQ ID NOS.: 1-27 and
B. Comparing the methylation status of the at least one cytosine to a threshold value for the biomarker, wherein the threshold value distinguishes between individuals with and without kidney cancer, wherein the comparison of the methylation status to the threshold value is predictive of the presence or absence of kidney cancer in the individual.

### Computer-Based Methods

The calculations for the methods described herein can involve computer-based calculations and tools. For example, a methylation level for a DNA region or a CpG loci can be compared by a computer to a threshold value, as described herein. The tools are advantageously provided in the form of computer programs that are executable by a general purpose computer system (referred to herein as a “host computer”) of conventional design. The host computer may be configured with many different hardware components and can be made in many dimensions and styles (e.g., desktop PC, laptop, tablet PC, handheld computer, server, workstation, mainframe). Standard components, such as monitors, keyboards, disk drives, CD and/or DVD drives, and the like, may be included. Where the host computer is attached to a network, the connections may be provided via any suitable transport media (e.g., wired, optical, and/or wireless media) and any suitable communication protocol (e.g., TCP/IP); the host computer may include suitable networking hardware (e.g., modem, Ethernet card, WiFi card). The host computer may implement any of a variety of operating systems, including UNIX, R, Linux, Microsoft Windows, MacOS, or any other operating system.

Computer code for implementing aspects of the present invention may be written in a variety of languages, including PERL, C, C++, Java, JavaScript, Python, VBScript, AWK, or any other scripting or programming language that can be executed on the host computer or that can be compiled to execute on the host computer. Code may also be written or distributed in low level languages such as assembler languages or machine languages.

The host computer system advantageously provides an interface via which the user controls operation of the tools. In the examples described herein, software tools are implemented as scripts (e.g., using PERL), execution of which can be initiated by a user from a standard command line interface of an operating system such as Linux or UNIX. Those skilled in the art will appreciate that commands can be adapted to the operating system as appropriate. In other embodiments, a graphical user interface may be provided, allowing the user to control operations using a pointing device. Thus, the present invention is not limited to any particular user interface.

Scripts or programs incorporating various features of the present invention may be encoded on various computer readable media for storage and/or transmission. Examples of suitable media include magnetic disk or tape, optical storage media such as compact disk (CD) or DVD (digital versatile disk), flash memory, and carrier signals adapted for transmission via wired, optical, and/or wireless networks conforming to a variety of protocols, including the Internet.

In a further aspect, the invention provides computer implemented methods for determining the presence or absence of cancer (including but not limited to kidney cancer) in an individual. In some embodiments, the methods comprise: receiving, at a host computer, a methylation value representing the methylation level of at least one cytosine within a DNA region in a sample from the individual where the DNA region is at least 90%, 91%, 92%, 93%, 94%, 95%, 96%, 97%, 98%, or 99% identical to, or comprises, a sequence is selected from the group consisting of SEQ ID NOS: 1-27; and comparing, in the host computer, the methylation level to a threshold value, wherein the threshold value distinguishes between individuals with and without cancer (including but not limited to kidney cancer), wherein the comparison of the methylation level to the threshold value is predictive of the presence or absence of cancer (including but not limited to kidney cancer) in the individual.

In some embodiments, the receiving step comprises receiving at least two methylation values, the two methylation values representing the methylation level of at least one cytosine biomarkers from two different DNA regions; and the comparing step comprises comparing the methylation values to one or more threshold value(s) wherein the threshold value distinguishes between individuals with and without cancer (including but not limited to kidney cancer), wherein the comparison of the methylation value to the threshold value is predictive of the presence or absence of cancer (including but not limited to cancers of the bladder, breast, cervix, colon, endometrium, esophagus, head and neck, liver, lung(s), ovaries, kidney, rectum, and thyroid, and melanoma) in the individual.

In another aspect, the invention provides computer program products for determining the presence or absence of cancer (including but not limited to kidney cancer), in an individual. In some embodiments, the computer readable products comprise: a computer readable medium encoded with program code, the program code including: program code for receiving a methylation value representing the methylation status of at least one cytosine within a DNA region in a sample from the individual where the DNA region is at least 90%, 91%, 92%, 93%, 94%, 95%, 96%, 97%, 98%, or 99% identical to, or comprises, a sequence selected from the group consisting of SEQ ID NOS: 1-27 and program code for comparing the methylation value to a threshold value, wherein the threshold value distinguishes between individuals with and without cancer (including but not limited to kidney cancer), wherein the comparison of the methylation value to the threshold value is predictive of the presence or absence of cancer (including but not limited to kidney cancer), in the individual.

### Materials and Methods

### Tissues/Nucleic Acid:

Kidney tissues used for this study were collected at Stanford University Medical Center with patient informed consent under an IRB-approved protocol. Tissue samples were removed from each kidney, flash-frozen, and stored at −80° C. Nucleic acid was extracted from the tissues using QIAGEN AllPrep DNA kit (QIAGEN).

### DNA Methylation Analysis Via Illumina Infinium HumanMethylation27:

Five hundred nanograms of DNA from each tissue was sodium bisulfite treated using the EZ-96 DNA Methylation Kit (Deep-well format, ZymoResearch) with the alternative incubation protocol for the Infinium Methylation Assay. DNA methylation levels were assayed using the Illumina Infinium HumanMethylation27 RevB Beadchip Kits (Illumina). We analyzed HumanMethylation27 array results using Illumina BeadStudio software with the Methylation Module v3.2. Any negative beta scores were converted to a zero and any beta scores with an associated detection P-value of >0.01 were converted to “NA” and filtered from analysis. To correct any array-by-array variation, we imputed all missing values with KNN Impute, followed by array batch normalization using the ComBat R-package. Previously imputed values were converted back to “NA” for all further analyses. CpGs with “NA” in greater than 10% of samples was removed from the data set. We also removed CpGs with questionable mapping or that included a SNP of >3% minor allele frequency within 15 bp of the assayed CpG to avoid potential variation in probe hybridization. After quality control and filtering, we had 26,148 CpGs assayed in both kidney tumor and benign adjacent tissues.

We used the glm command with family set to binomial to perform logistic regression of possible combinations of the diagnostic biomarkers. We selected our best model based on a maximum ROC curve area and a minimum AIC value.

Discovery of CpG Loci with DNA Methylation Levels Determinative of Kidney Cancer:

We performed PamR (version 1.54) analysis on all filtered CpGs as described in the PamR manual with RStudio (version 0.97.551) in R (version 3.0.0). Based on visual examination of the training errors and cross-validation results, we minimized the miss-rate and set the shrinkage threshold to 10.74 for all tumor and benign adjacent normal classification, and 14.8 for clear cell tumor and benign adjacent normal classification.

### Logistic Regression and Receiver Operating Characteristic (ROC) Curves:

After the CpGs were identified using PamR, we used logistic regression to determine the predictive power of these CpGs for kidney cancer diagnosis. We used the glm command with family set to binomial to perform logistic regression of possible combinations of the diagnostic biomarkers. We selected our best model based on a maximum ROC curve area and a minimum AIC value, selecting a four CpG model for ccRCC diagnosis and a five CpG model for diagnosis of RCC across multiple subtypes.

We used the sensitivity and specificity to produce ROC curves for these models. Since a perfect predictor will have an area under the ROC curve of 1, we then calculated the area under the ROC curves. The best ccRCC model had an area of 0.990 and the best multiple subtype model had an area of 0.991. To test the ability of the CpGs to predict recurrence we randomly selected CpGs that were not identified using linear regression. Using these CpGs we developed logistic regression models, the ROC curves, and calculated the area under these curves. For these models the area was close to 0.5, which is the expected area when a model provides no predicative power.

### Validation in the Cancer Genome Atlas Datasets

We downloaded TCGA Illumina results for all kidney cancer patients. Diagnostic biomarker validation for ccRCC patients utilized HumanMethylation27 tumor and matched benign adjacent normal ccRCC TCGA data only (ROC area is 0.972). Diagnostic biomarker validation for the general RCC patients utilized both HumanMethylation27 and HumanMethylation450 tumor and matched benign adjacent normal ccRCC, pRCC, and ChRCC TCGA data (ROC area is 0.990).

TABLE 2

| | | SEQ |
|---|---|---|
| CpG Loci | Nucleotide Sequence | ID. NO. |
| cg02706881 | CGCACAGATGTGCTGTTCTAACTTGGGATAAATGTGGATCTCGTGAATCC | SEQ ID NO. 1 |
| cg03562120 | TGCCTGGGAGTGACCTCACAGCTGCCGGAACATAAAGACTCACAGGTCCG | SEQ ID NO. 2 |
| cg04511534 | CAAGTCCTGGTGCAGGAGGCACCTGCTGGGCAGGTTGGGGCCTGACTACG | SEQ ID NO. 3 |
| cg04598121 | AGGAGCCCGGGGCCGAGCAACAGCAGCCAAGTGCAAAGTGTCAGGAACCG | SEQ ID NO. 4 |
| cg04988978 | CTTTTGACTGAATCAGTCTACCTCTCTGGGCCCTGGTCAGGCTGAGCTCG | SEQ ID NO. 5 |
| cg05379350 | TGTATGTGTCACACTCTTGCTGAATACGCCCACTGCTAACAATATGGACG | SEQ ID NO. 6 |
| cg06130787 | CGCCCACTCTGTGGCCGTGAGTGAGCTCTGTGTGTGTCCCAGTGACTAGC | SEQ ID NO. 7 |
| cg08749917 | CGGTCTAAAAATCCTCATCGACAAGACCAGGAGGAAGCAGGACCCAGCTC | SEQ ID NO. 8 |
| cg10045881 | GCTTCTTCTGGGATACACATTCTCTAGGTCTTTTATCCACTGAGGTTTCG | SEQ ID NO. 9 |
| cg11098259 | CGGGCCCTGGTCCAGAAAAGATTTTCATGTTACACAATTGCAGGCTTCTG | SEQ ID NO. 10 |
| cg12782180 | GGGGGTGGCTGTGAGGGGCTCCGCGGAGCGGGCTGGGGCATACGGCTGCG | SEQ ID NO. 11 |
| cg12907644 | ACAAACTGGTCTAAGACAAGTTCCTGGATGCCGGTGGTTTCTTCATCCCG | SEQ ID NO. 12 |
| cg12939547 | AGATAAGGTGGGCAACAGTCAATCCAAAGGGCCTCCCTGGAGCCCCGTCG | SEQ ID NO. 13 |
| cg13156411 | CGGGCATGTCTTGTCTGCCCCATAGCACGGCCCAGGTATTTAGACACTCA | SEQ ID NO. 14 |
| cg14370448 | CGCCACTGGCTTCCCGCCACCCGAAGGGAGCTCTGGACCCTCAGAGCCCC | SEQ ID NO. 15 |
| cg14391855 | CGGCCTCAGTCCCCACAGGCCCCAGCCATGCTCTGGGGGCACCTTTGGCT | SEQ ID NO. 16 |
| cg14456683 | GCTTTACAATACCTGGGATTGATGAGGCGGGCGGGCCAATGAGCTGCGCG | SEQ ID NO. 17 |
| cg15484375 | ACAAAACGGTCTAAGACAAGTTCCTGGATGCCAGTGGTTTCTTCATCCCG | SEQ ID NO. 18 |
| cg16592658 | CGCATGTCTGTGTAGCTATGTCTGTGTAGCTCTATGGATACCTCTGAGCT | SEQ ID NO. 19 |
| cg17568996 | CGACAACCAGCAAATCCCCAGAGACAGGTCCCTGGGAATTAGCTGCGCCG | SEQ ID NO. 20 |
| cg18003231 | GGCTCATCAGTTTGGGGACTGGCTTCATCGCTTGTTCTGTCCAGCAGTCG | SEQ ID NO. 21 |
| cg22628873 | GGTTCGTAACTCCCTGTGCGTGTTTTGCGACTCTTGTCCAGAAGGTAGCG | SEQ ID NO. 22 |
| cg22719623 | CAAGTTGACCCAGGAACCGGGGCTGGGTGCTGGGGAGCAACTTGAGTACG | SEQ ID NO. 23 |
| cg23320056 | ACTGCGTTACCTCAGTCTTTAAAGACCCGCAGGCAGGAGAATTCCATCCG | SEQ ID NO. 24 |
| cg26366091 | AAGTTTCACAAGTCTGCCAGGGGAAGTCCCTGGACTTCTTGCTTCTTTCG | SEQ ID NO. 25 |
| cg26514492 | CGAGGCCATGCTGTCATCACCAGTAAGATACCCCAGCCCGGTTGGCTAAC | SEQ ID NO. 26 |
| cg26954174 | CGTGTGAGCCATACACACCCCAGCTAGTGACGTTGGGCTTCTGTGGACAC | SEQ ID NO. 27 |

The sequences of the CpG loci described herein. The actual methylation
site is underlined in the nucleotide sequence.

We claim:

1. A method for determining the presence or absence of kidney cancer in an individual, the method comprising:
a. identifying an individual in need of the prevention or treatment of kidney cancer;
b. obtaining a biological sample from the individual and isolating the DNA therefrom;
c. determining the methylation level of at least one cytosine within a DNA region in a sample from the individual where the DNA region is at least 90% identical to a sequence selected from the group consisting of SEQ ID NOS: 1, 4, 6, 7, 8, 11, 12, 13, 14, 17, 20, 21, 22, 23, and 24; and
d. comparing the methylation level of the at least one cytosine to a threshold value for the at least one cytosine, wherein the threshold value distinguishes between individuals with and without kidney cancer, wherein the comparison of the methylation level to the threshold value is predictive of the presence or absence of kidney cancer in the individual.

2. The method of claim 1 wherein said sample is a biopsy sample.

3. The method of claim 1 wherein said sample is a blood sample.

4. The method of claim 1 wherein said sample is a urine sample.

5. The method of claim 1 wherein the methylation level of at least 3 DNA regions are determined.

6. The method of claim 1 wherein the methylation level of at least 5 DNA regions are determined.

7. A kit for determining the presence or absence of kidney cancer in an individual, the kit comprising:
a. a plurality of nucleic acid primers configured to bind to a nucleic acid at least 90% identical to a sequence selected from the group consisting of SEQ ID NOS.: 1, 4, 6, 7, 8, 11, 12, 13, 14, 17, 20, 21, 22, 23, and 24;
b. wherein the primers are for use in a polymerase chain reaction (PCR) reaction;
wherein the primers are configured to aid in the determination of the methylation level of at least one cytosine within the nucleic acid.

8. The method of claim 7 wherein the nucleic acid is at least 92% identical to a sequence selected from the group consisting of SEQ ID NOS.: 1, 4, 6, 7, 8, 11, 12, 13, 14, 17, 20, 21, 22, 23, and 24.

9. The method of claim 7 wherein the nucleic acid is at least 95% identical to a sequence selected from the group consisting of SEQ ID NOS.: 1, 4, 6, 7, 8, 11, 12, 13, 14, 17, 20, 21, 22, 23, and 24.

10. The method of claim 7 wherein the methylation level of at least 3 nucleic acids is determined.

11. A method for determining the presence or absence of clear cell renal cell carcinoma in an individual, the method comprising:
a. identifying an individual in need of the prevention or treatment of kidney cancer;
b. obtaining a biological sample from the individual and isolating the DNA therefrom;
c. determining the methylation level of at least one cytosine within a DNA region in a sample from the individual where the DNA region is at least 90% identical to a sequence selected from the group consisting of SEQ ID NOS: 2, 9, 10, 15, 19, 25, 26 and 27; and
d. comparing the methylation level of the at least one cytosine to a threshold value for the at least one cytosine, wherein the threshold value distinguishes between individuals with and without kidney cancer, wherein the comparison of the methylation level to the threshold value is predictive of the presence or absence of kidney cancer in the individual.

12. The method of claim 11 wherein said sample is a biopsy sample.

13. The method of claim 11 wherein said sample is a blood sample.

14. The method of claim 11 wherein said sample is a urine sample.

15. The method of claim 11 wherein the methylation level of at least 3 DNA regions are determined.

16. A kit for determining the presence or absence of clear cell renal cell carcinoma in an individual, the kit comprising:
a. a plurality of nucleic acid primers configured to bind to a nucleic acid at least 90% identical to a sequence selected from the group consisting of SEQ ID NOS.: 2, 9, 10, 15, 19, 25, 26 and 27;
b. wherein the primers are for use in a polymerase chain reaction (PCR) reaction;
wherein the primers are configured to aid in the determination of the methylation level of at least one cytosine within the nucleic acid.

17. The method of claim 7 wherein the nucleic acid is at least 92% identical to a sequence selected from the group consisting of SEQ ID NOS.: 2, 9, 10, 15, 19, 25, 26 and 27.

18. The method of claim 7 wherein the nucleic acid is at least 95% identical to a sequence selected from the group consisting of SEQ ID NOS.: 2, 9, 10, 15, 19, 25, 26 and 27.

19. A method for determining the presence or absence of at least one of kidney cancer or clear cell renal cell carcinoma in an individual, the method comprising:
a. identifying an individual in need of the prevention or treatment of kidney cancer;
b. obtaining a biological sample from the individual and isolating the DNA therefrom;
c. determining the methylation level of at least one cytosine within a DNA region in a sample from the individual where the DNA region is at least 90% identical to a sequence selected from the group consisting of SEQ ID NOS: 3, 5, 16 and 18; and
d. comparing the methylation level of the at least one cytosine to a threshold value for the at least one cytosine, wherein the threshold value distinguishes between individuals with and without kidney cancer, wherein the comparison of the methylation level to the threshold value is predictive of the presence or absence of kidney cancer in the individual.


SEQUENCE LISTING (27 sequences)

SEQ ID NO: 1 - DNA, 50 bp, Homo sapiens
cgcacagatgtgctgttctaacttgggataaatgtggatctcgtgaatcc

SEQ ID NO: 2 - DNA, 50 bp, Homo sapiens
tgcctgggagtgacctcacagctgccggaacataaagactcacaggtccg

SEQ ID NO: 3 - DNA, 50 bp, Homo sapiens
caagtcctggtgcaggaggcacctgctgggcaggttggggcctgactacg

SEQ ID NO: 4 - DNA, 50 bp, Homo sapiens
aggagcccggggccgagcaacagcagccaagtgcaaagtgtcaggaaccg

SEQ ID NO: 5 - DNA, 50 bp, Homo sapiens
cttttgactgaatcagtctacctctctgggccctggtcaggctgagctcg

SEQ ID NO: 6 - DNA, 50 bp, Homo sapiens
tgtatgtgtcacactcttgctgaatacgcccactgctaacaatatggacg

SEQ ID NO: 7 - DNA, 50 bp, Homo sapiens
cgcccactctgtggccgtgagtgagctctgtgtgtgtcccagtgactagc

SEQ ID NO: 8 - DNA, 50 bp, Homo sapiens
cggtctaaaaatcctcatcgacaagaccaggaggaagcaggacccagctc

SEQ ID NO: 9 - DNA, 50 bp, Homo sapiens
gcttcttctgggatacacattctctaggtcttttatccactgaggtttcg

SEQ ID NO: 10 - DNA, 50 bp, Homo sapiens
cgggccctggtccagaaaagattttcatgttacacaattgcaggcttctg

SEQ ID NO: 11 - DNA, 50 bp, Homo sapiens
gggggtggctgtgaggggctccgcggagcgggctggggcatacggctgcg

SEQ ID NO: 12 - DNA, 50 bp, Homo sapiens
acaaactggtctaagacaagttcctggatgccggtggtttcttcatcccg

SEQ ID NO: 13 - DNA, 50 bp, Homo sapiens
agataaggtgggcaacagtcaatccaaagggcctccctggagccccgtcg

SEQ ID NO: 14 - DNA, 50 bp, Homo sapiens
cgggcatgtcttgtctgccccatagcacggcccaggtatttagacactca

SEQ ID NO: 15 - DNA, 50 bp, Homo sapiens
cgccactggcttcccgccacccgaagggagctctggaccctcagagcccc

SEQ ID NO: 16 - DNA, 50 bp, Homo sapiens
cggcctcagtccccacaggccccagccatgctctgggggcacctttggct

SEQ ID NO: 17 - DNA, 50 bp, Homo sapiens
gctttacaatacctgggattgatgaggcgggcgggccaatgagctgcgcg

SEQ ID NO: 18 - DNA, 50 bp, Homo sapiens
acaaaacggtctaagacaagttcctggatgccagtggtttcttcatcccg

SEQ ID NO: 19 - DNA, 50 bp, Homo sapiens
cgcatgtctgtgtagctatgtctgtgtagctctatggatacctctgagct

SEQ ID NO: 20 - DNA, 50 bp, Homo sapiens
cgacaaccagcaaatccccagagacaggtccctgggaattagctgcgccg

SEQ ID NO: 21 - DNA, 50 bp, Homo sapiens
ggctcatcagtttggggactggcttcatcgcttgttctgtccagcagtcg

SEQ ID NO: 22 - DNA, 50 bp, Homo sapiens
ggttcgtaactccctgtgcgtgttttgcgactcttgtccagaaggtagcg

SEQ ID NO: 23 - DNA, 50 bp, Homo sapiens
caagttgacccaggaaccggggctgggtgctggggagcaacttgagtacg

SEQ ID NO: 24 - DNA, 50 bp, Homo sapiens
actgcgttacctcagtctttaaagacccgcaggcaggagaattccatccg

SEQ ID NO: 25 - DNA, 50 bp, Homo sapiens
aagtttcacaagtctgccaggggaagtccctggacttcttgcttctttcg

SEQ ID NO: 26 - DNA, 50 bp, Homo sapiens
cgaggccatgctgtcatcaccagtaagataccccagcccggttggctaac

SEQ ID NO: 27 - DNA, 50 bp, Homo sapiens
cgtgtgagccatacacaccccagctagtgacgttgggcttctgtggacac